(19) United States
(12) Patent Application Publication
(10) Pub. No.: US 2007/0219613 A1
(43) Pub. Date: Sep. 20, 2007

(54) APPARATUS AND METHODS FOR INTERLOCKING STENT SEGMENTS

(75) Inventors: Stephen Kao, Sunnyvale, CA (US); Jeremy Dittmer, Palo Alto, CA (US); David Lowe, Redwood City, CA (US)

Correspondence Address: LEVINE BAGADE HAN LLP, 2483 EAST BAYSHORE ROAD, SUITE 100, PALO ALTO, CA, 94303, US

(73) Assignee: Xtent, Inc., Menlo Park, CA (US)

(21) Appl. No.: 11/687,903

(22) Filed: Mar. 19, 2007

Related U.S. Application Data
(60) Provisional application No. 60/784,309, filed on Mar. 20, 2006.

(51) Int. Cl. A61F 2/84; A61F 2/90

(52) U.S. Cl. 623/1.11; 623/1.16

(57) ABSTRACT

Apparatus and methods for interlocking stent segments which provide for a secure engagement between the expanded stent segments are described herein. Stent segments which are able to slide freely relative to one another along the deployment catheter prior to expansion may be secured to one another when expanded and/or deployed into the vessel. Securement upon expansion of the stent segments may be accomplished, in part, by utilizing one or more coupling mechanisms between adjacent stent segments which securely interlock the segments to one another by taking advantage of the changing geometry of the stents during expansion.

### CROSS-REFERENCE TO RELATED APPLICATIONS

This application claims the benefit of priority to U.S. Provisional Patent Application No. 60/784,309 filed Mar. 20, 2006, which is incorporated herein by reference in its entirety.

### FIELD OF THE INVENTION

The present invention relates generally to medical devices and methods. More specifically, the present invention relates to apparatus and methods for deploying a luminal prosthesis which may have one or more linked or otherwise coupled segments.

### BACKGROUND OF THE INVENTION

Stenting is an important treatment option for patients with coronary artery disease and has become a common medical procedure. The procedure is mainly directed at revascularization of stenotic vessels where a blocked artery is dilated and a stent is placed in the vessel to help maintain luminal patency. The stent is a small, tubular shaped device that can be expanded in a diseased vessel, thereby providing support to the vessel wall which in turn helps to maintain luminal patency.

Restenosis, where treated vessels such as coronary arteries tend to become re-occluded following stent implantation, was a problem in early stent technology. However, recent improvements in stent design, delivery systems and techniques along with the development of drug eluting stents have significantly reduced restenosis rates. Because of the improved efficacy of stenting, the number of stenting procedures has dramatically increased worldwide.

A balloon expandable stent is delivered to the coronary arteries using a long, flexible vascular catheter with a balloon on the distal end over which the stent is mounted. The delivery catheter is introduced into the vascular system percutaneously through a femoral or radial artery. Once the stent is delivered to the target treatment site, the delivery catheter balloon is expanded which correspondingly expands and permanently deforms the stent to a desired diameter. The balloon is then deflated and removed from the vessel, leaving the stent implanted in the vessel at the lesion site.

Self-expanding stents are another variation of luminal prosthesis where the stent is constrained during delivery and then released at a desired location. When the stent is released from the constraining mechanism, the stent resiliently expands into engagement with the vessel wall. The delivery catheter is then removed and the stent remains in its deployed position.

With current stents lesion size must be assessed in order to determine the appropriate stent length required to effectively cover the lesion. Fluoroscopy and angiography are therefore used to evaluate the lesion prior to stent delivery. A stent of appropriate size is then delivered to the lesion. Sometimes, however, lesion length cannot be assessed accurately and can result in the selection of stents which are not long enough to adequately cover the target lesion. To address this shortfall, an additional stent must be delivered adjacent to the initially placed stent. When lesion length requires multiple stents to be delivered, multiple delivery catheters are required since typically only one stent is provided with each delivery catheter. The use of multiple delivery catheters results in greater cost and longer procedure time. In addition, and particularly in peripheral stenting, overlapping of stents can be problematic. To overcome this shortcoming, recent stent delivery systems have been designed to streamline this process by allowing multiple stent segments to be delivered simultaneously from a single delivery catheter, thereby permitting customization of stent length in situ to match the size of lesion being treated.

Various designs have been proposed for custom length prostheses such as those described in U.S. patent application Ser. No. 10/306,813 filed Nov. 27, 2002 (U.S. Patent Publication 2003-0135266 A1) which is incorporated herein by reference. These designs utilize delivery systems pre-loaded with multiple stent segments, of which some or all of the stent segments can be delivered to the site of a lesion. This allows the length of the prosthesis to be customized to match the lesion size more accurately.

Having a delivery system pre-loaded with multiple stent segments which are unconnected to one another allows for a catheter system which can retain its flexibility, particularly during advancement and maneuvering along tortuous intravascular pathways. Although these stent segments may be individually deployed or expanded against a lesion such that the stents are expanded and positioned next to one another but unconnected, it may be desirable for these deployed stent segments to be connected or otherwise coupled to one another in their expanded configurations.

Having the expanded stent segments connected to one another may help to ensure that the deployed stent segments are secured with respect to one another and along the vessel wall. Coupling between adjacent stent segments may additionally help to ensure that there are no gaps between each adjacent stent segment and may also help to prevent any migration of individual stent segments along the vessel walls.

In situations where vessels are tapered or have other irregularities in diameter, e.g., around the ostia of a vessel, a single balloon of constant diameter may have difficulty in expanding all of the stent segments to engage tightly with the vessel wall. Accordingly, stent delivery systems and methods are desired which can accommodate tapered and irregularly sized vessels while minimizing or preventing a stent segment from moving, dislodging or tilting in the vessel following deployment. Additionally, such a stent delivery system is desired which can deliver one or more stent segments which are uncoupled from one another so as to maintain a flexibility of the system but which can then couple or secure the one or more stent segments when deployed into a vessel. Such stent systems should also permit stent length customization in situ and allow treatment of multiple lesions of various sizes, without requiring removal of the delivery catheter from the patient.

### SUMMARY OF THE INVENTION

As described, customized, variable length, luminal medical prosthesis can be delivered effectively to one or more treatment sites in irregularly shaped or highly tapered coronary arteries or other vessels, using a single delivery device, during a single interventional procedure. Because the length of the deployed stent is variable depending upon the length of the lesion to be treated, the number of stent segments deployed into a vessel may be altered in situ. Additionally, one or more disconnected stent segments enable a delivery catheter to maintain its flexibility, particularly when the delivery catheter is advanced through tortuous intravascular pathways. Thus, ease of release and deployment of stent segments adjacent to one another is desirable.

However, it is desirable to maintain a relatively secure engagement between the expanded stent segments and the vessel wall, particularly along tapered vessels or vessels with an uneven anatomy, while also minimizing or preventing migration of an expanded stent segment relative to the other expanded stent segments. Stent segments which are able to slide freely relative to one another along the deployment catheter prior to expansion may be secured to one another when expanded and/or deployed into the vessel. Securement upon expansion of the stent segments may be accomplished, in part, by utilizing one or more coupling mechanisms between adjacent stent segments which securely interlock the segments to one another by taking advantage of the changing geometry of the stents during expansion.

One method for delivering a luminal prosthesis to at least one treatment site comprises providing a plurality of radially expandable prosthetic stent segments arranged axially along a delivery catheter with at least some of the adjacent prosthetic stent segments being disconnected from one another and having one or more coupling structures between the prosthetic stent segments. The delivery catheter may be positioned at a first treatment site and two or more prosthetic stent segments are selected for deployment. The selected segments are radially expanded without expanding the segments remaining on the delivery catheter and one or more coupling mechanisms between the expanded stent segments may permit the selected stent segments to become secured to one another in their expanded state.

Stent delivery systems and methods may be used to stent body lumens such as blood vessels and coronary arteries in particular. The systems and methods are also used frequently in the peripheral vascular and cerebral vascular systems as well as other body ducts such as the biliary duct, fallopian tubes and the like. Additional uses may also include applications in orthopedic, cardiac, valvular and other prostheses.

Variations of the coupling mechanisms which interlock the expanding stent segments may include a coupling structure extending axially between adjacent prosthetic stent segment ends that is movable between an open position and a pinched or closed position which permits prosthetic stent segments to be coupled together when the coupling structure is moved, upon deployment. The coupling structures may be moved by deformation or movement of the struts of the stent segments to which they are attached.

For example, the coupling structure may comprise a pair of jaw members which engage onto a projection upon expansion of the prosthetic stent segments. These jaw members may be configured into various geometries, such as parallel projections, curved projections, etc. The coupling member which is grasped or pinched upon by the adjacent jaw members may comprise a T-shaped member or variations thereof. Expansion of the stent segments causes the jaw members to close and engage the coupling member.

In yet other variations, the stent segments may be loosely connected during delivery to maintain a flexibility of the catheter and positioning of the stent segments in situ. When expanded, the adjacent stent segments may become stiffened to more securely couple the respective stent segments to one another. Additionally, the stent segments and coupling mechanisms may be configured such that when expanded and secured to one another, a shape or bias may be imparted to the luminal prosthesis, e.g., a curve.

### BRIEF DESCRIPTION OF THE DRAWINGS

FIG. 1 shows a perspective view of a luminal prosthesis delivery system composed of multiple linked stents arranged axially along a delivery catheter.

FIG. 2A is a side view of a luminal prosthesis delivery system with multiple linked stents mounted on a delivery catheter and positioned in a vessel, at a target lesion site.

FIG. 2B is a side view illustrating a group of stent segments selected for deployment.

FIG. 2C is a side view illustrating the stent segments selected for deployment separated from the remaining stent segments.

FIG. 2D is a side view illustrating the selected stent segments being radially expanded while the remaining stent segments are left behind on the delivery catheter.

FIG. 3A shows one variation of a coupling mechanism for engaging adjacent stent segments with a T-shaped member engaging a set of jaws.

FIG. 3B illustrates the coupling mechanism in FIG. 3A with the jaws enclosed at least partially around the T-shaped member upon expansion or deployment of the stent segments.

FIG. 4A shows another variation of a coupling mechanism between adjacent stent segments with adjacent stent segments shown unrolled and flattened.

FIG. 4B is a detailed view of the coupling mechanism of FIG. 4A having projecting members which are keyed to be received in a secure manner within a corresponding receiving channel in the adjacent stent segment.

FIG. 4C shows the coupling mechanism of FIG. 4A-4B in the engaged position once the stent segments have been expanded or deployed.

FIG. 5A shows another variation of a coupling mechanism between adjacent stent segments with adjacent stent segments shown unrolled and flattened.

FIG. 5B is a detailed view of the coupling mechanism of FIG. 5A utilizing a pinching mechanism for engaging a projection extending from the adjacent stent segment.

FIG. 5C shows the coupling mechanism of FIG. 5B in the engaged position once the stent segments have been expanded or deployed.

FIG. 6A shows another variation of a coupling mechanism between adjacent stent segments with adjacent stent segments shown unrolled and flattened.

FIG. 6B illustrates a detailed view of the coupling mechanism of FIG. 6A utilizing a curved pinching or jaw mechanism for engaging a projection extending from the adjacent stent segment.

FIG. 6C illustrates the coupling mechanism of FIG. 6B in the engaged position once the stent segments have been expanded or deployed.

FIG. 6D illustrates a variation of the curved jaw mechanism with the jaw members projected at an angle relative to an axis of the coupling mechanism.

FIG. 6E illustrates another variation of the curved jaw mechanism with the jaw members projected parallel relative to one another.

FIG. 7A shows another variation of a coupling mechanism between adjacent stent segments with adjacent stent segments shown unrolled and flattened.

FIG. 7B shows a detailed view of the coupling mechanism of FIG. 7A utilizing pinching members which project parallel relative to one another.

FIG. 7C shows the coupling mechanism of FIG. 7B in the engaged position once the stent segments have been expanded or deployed.

FIG. 8A shows a detailed side view of an alternative coupling member which is rounded to facilitate its release and engagement between adjacent stent segments.

FIG. 8B shows a detailed side view of another alternative coupling member which is configured with one or more intra-stent cusps which facilitate and maintain a sufficient separation distance between adjacent stent segments.

FIGS. 8C and 8D illustrate the coupling member of FIG. 8B prior to and after engagement with the adjacent stent, respectively.

FIG. 9A illustrates yet another variation of a coupling mechanism between adjacent stent segments where pinching or grasping members are alternated between coupling members.

FIG. 9B illustrates another variation where pinching or grasping members are alternated in groups between coupling members.

FIG. 10 shows yet another variation which utilizes one or more single arms or coupling members which extend between adjacent stent segments.

FIGS. 11A and 11B illustrate, respectively, the variation of FIG. 10 where a stent segment may utilize at least two arms positioned along each side of a stent segment and which swings into and locks against an opposing arm on an adjacent stent segment when expanded.

FIG. 12A shows another variation of a coupling mechanism between adjacent stent segments with adjacent stent segments shown unrolled and flattened.

FIGS. 12B and 12C show detailed views of the coupling mechanism of FIG. 12A having projecting members which pinch against one another in their secured configuration.

FIG. 13A shows another variation with adjacent stent segments having coupling mechanisms aligned in alternating pairs.

FIG. 13B shows a perspective view of the coupled stent segments of FIG. 13A.

FIGS. 14A and 14B show unexpanded stent segments uncoupled from one another.

FIG. 14C illustrates the expanded stent segments from FIGS. 14A and 14B with the coupling mechanism engaged to impart a shape to the stent assembly.

### DETAILED DESCRIPTION OF THE INVENTION

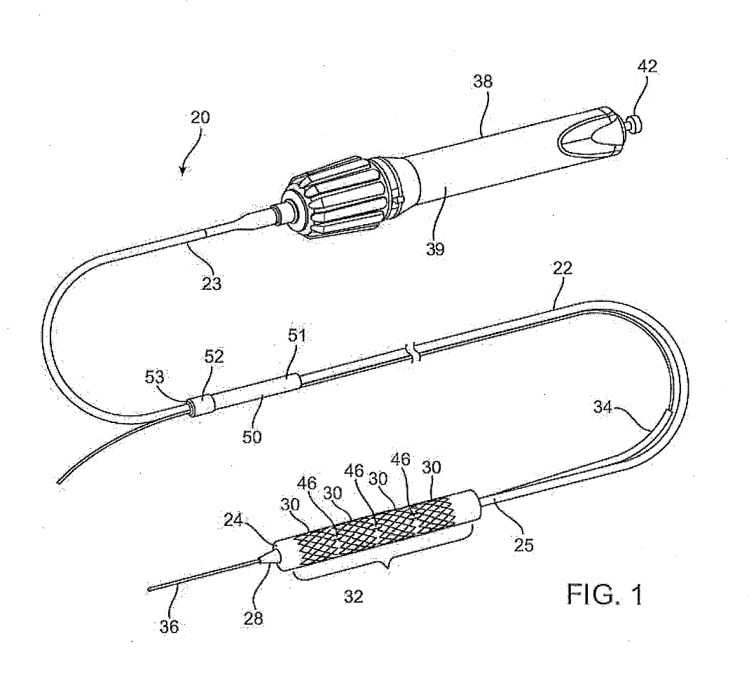

An example of a luminal prosthesis delivery system 20 which may be utilized with the one or more stent segments described herein is illustrated in the perspective assembly view of FIG. 1. Luminal prosthesis delivery system 20 generally comprises a catheter shaft 22 with an outer sheath 25 slidably disposed over an inner shaft (not shown). An inflatable balloon 24 is mounted on the inner shaft and is exposed by retracting sheath 25 relative to the inner shaft. A tapered nosecone 28, composed of a soft elastomeric material to reduce trauma to the vessel during advancement of the delivery system is attached distally of the inflatable balloon 24. A luminal prosthesis 32 comprises a plurality of separable stent segments 30 mounted over the inflatable balloon 24 for expansion. A guidewire tube 34 is slidably positioned through sheath 25 proximal to the inflatable balloon 24. A guidewire 36 is positioned slidably through guidewire tube 34, inflatable balloon 24 and nosecone 28 and extends distally thereof.

A handle 38 is attached to a proximal end 23 of the sheath 25. The handle performs several functions, including operating and controlling the catheter body 22 and the components in the catheter body. Various embodiments of the handle 38 along with details concerning its structure and operation are described in U.S. patent application Ser. No. 10/746,466 filed Dec. 23, 2003 (U.S. Patent Publication 2005-0149159 A1), the full disclosure of which is hereby incorporated by reference.

Handle 38 includes a housing 39 which encloses the internal components of the handle 38. The inner shaft is preferably fixed to the handle, while the outer sheath 25 is able to be retracted and advanced relative to handle 38. An adaptor 42 is attached to handle 38 at its proximal end and is fluidly coupled to the inner shaft in the interior of the housing of handle 38. The adaptor 42, e.g., which may be a luer connector, is configured to be fluidly coupled with an inflation device which may be any commercially available balloon inflation device such as those sold under the trade name INDEFLATOR™ manufactured by Abbot (formerly Guidant Corporation of Santa Clara, Calif.). The adaptor is in fluid communication with the inflatable balloon 24 via an inflation lumen in the inner shaft (not shown) to permit inflation of the inflatable balloon 24.

The outer sheath 25 and guidewire 36 each extend through a slider assembly 50 located on the catheter body 22 at a point between its proximal and distal ends. The slider assembly 50 is adapted for insertion into and sealing with a hemostasis valve, such as on an introducer sheath or guiding catheter, while still allowing relative movement of the outer sheath 25 relative to the slider assembly 50. The slider assembly 50 includes a slider tube 51, a slider body 52, and a slider cap 53.

The outer sheath 25 may be composed of any of a variety of biocompatabile materials, such as but not limited to, a polymer such as PTFE, FEP, polyimide, or PEBAX® (Arkema France Corp., France), may be reinforced with a metallic or polymeric braid to resist radial expansion of inflatable balloon 24, and/or the like. Inflatable balloon 24 may be formed of a compliant or semi-compliant polymer such as PEBAX®, Nylon, polyurethane, polypropylene, PTFE or other suitable polymer. Compliance of the polymer may be adjusted to provide optimal inflation and stent expansion. Additional aspects of the luminal prosthesis delivery system are described in U.S. patent application Ser. No. 10/306,813 filed Nov. 27, 2002 (U.S. Patent Publication 2003-0135266 A1); U.S. patent application Ser. No. 10/637,713 filed Aug. 8, 2003 (U.S. Patent Publication 2004-0098081 A1); U.S. patent application Ser. No. 10/738,666 filed Dec. 16, 2003 (U.S. Patent Publication 2004-0186551 A1); U.S. patent application Ser. No. 11/104,305 filed Apr. 11, 2005 (U.S. Patent Publication 2006-0229700 A1); and U.S. application Ser. No. 11/148,545 filed Jun. 8, 2005 (U.S. Patent Publication 2006-0282147 A1), the full disclosures of which are hereby incorporated by reference.

The luminal prosthesis 32 may be composed of one or more prosthetic stent segments 30 which are disposed over an inflation balloon 24. Each stent segment may range from about 2-30 mm in length, more typically about 2-20 mm in length, and preferably being about 2-10 mm in length and less than 7 mm in additional preferred embodiments. Usually 2-50, more typically 2-25 and preferably 2-10 stent segments 30 may be positioned axially over the inflation balloon 24 and the inflation balloon 24 has a length suitable to accommodate the number of stent segments. Stent segments 30 may be positioned in direct contact with an adjacent stent segment or a space may exist in between segments. One or more coupling elements 46 may link the adjacent stent segments 30 together, as described in further detail below. Furthermore, the stent segments 30 may be deployed individually or in groups of two or more at one or multiple treatment sites within the vessel lumen.

Prosthetic stent segments 30 may be composed of a malleable metal so they may be plastically deformed by inflation balloon 24 as they are radially expanded to a desired diameter in the vessel at the target treatment site. The stent segments 30 may also be composed of an elastic or superelastic shape memory alloy such as Nitinol so that the stent segments 30 self-expand upon release into a vessel by retraction of the outer sheath 25. In this case, an inflation balloon 24 is not required but may still be used for pre- and/or post-dilatation of a lesion or augmenting expansion of the self-expanding stent segments. Other materials such as biocompatible polymers may be used to fabricate prosthetic stent segments and these materials may further have bioabsorbable or bioerodable properties.

Stent segments 30 may have any of a variety of common constructions, such as but not limited to those described in U.S. patent application Ser. No. 10/738,666 filed Dec. 16, 2003, which was previously incorporated by reference. Constructions may include, for example, closed cell constructions including expansible ovals, ellipses, box structures, expandable diamond structures, etc. In addition, the closed cells may have complex slotted geometries such as H-shaped slots, I-shaped slots, J-shaped slots, etc. Suitable open cell structures include zigzag structures, serpentine structures, and the like. Such conventional stent structures are well described in the patent and medical literature. Specific examples of suitable stent structures are described in the following U.S. patents, the full disclosures of which are incorporated herein by reference: U.S. Pat. No. 6,315,794; U.S. Pat. No. 5,980,552; U.S. Pat. No. 5,836,964; U.S. Pat. No. 5,421,955; and U.S. Pat. No. 4,776,337.

Moreover, prosthetic stent segments 30 may be coated, impregnated, infused or otherwise coupled with one or more drugs that inhibit restenosis, such as Rapamycin, Everolimus, Paclitaxel, analogs, prodrugs, or derivatives of the aforementioned such as Biolimus A9® (Biosensors International), or other suitable agents, preferably carried in a durable or bioerodable polymeric carrier. Alternatively, stent segments 30 may be coated with other types of drugs or therapeutic materials such as antibiotics, thrombolytics, anti-thrombotics, anti-inflammatories, cytotoxic agents, anti-proliferative agents, endothelial cell attractors or promoters, vasodilators, gene therapy agents, radioactive agents, immunosuppressants, chemotherapeutics and/or stem cells, or combinations thereof. Such materials may be coated over all or a portion of the surface of stent segments 30, or stent segments 30 may include apertures, holes, channels, or other features in which such materials may be deposited.

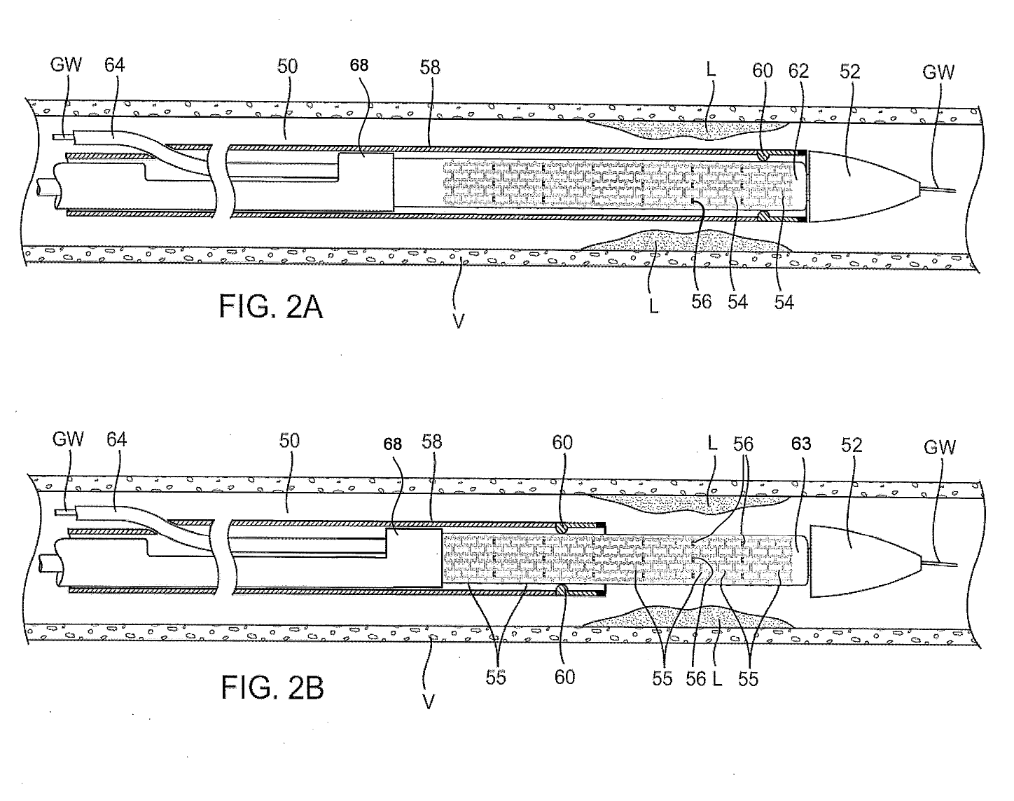

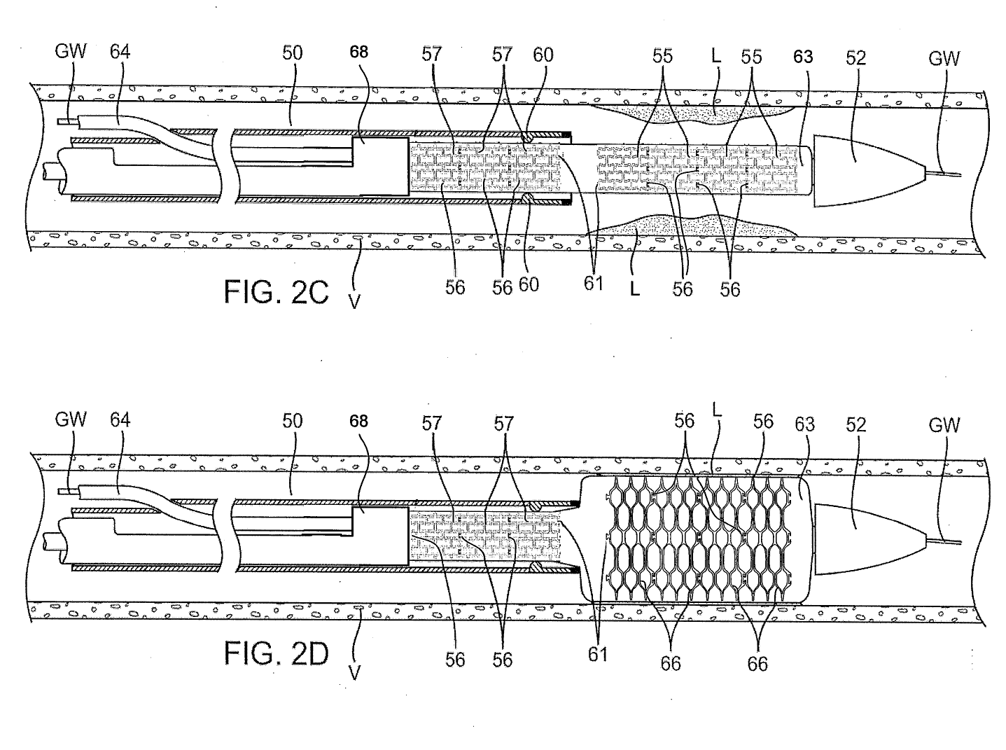

FIGS. 2A to 2D illustrate one example for delivering one or more stent segments in a vessel utilizing the delivery system described above. In FIG. 2A, a partial cross-sectional side view of a luminal prosthesis delivery system 50 is introduced into a vessel V and advanced to the site of a lesion L. The delivery system 50 may have multiple stent segments 54 mounted over a delivery catheter 62 with one or more of the stent segments 54 having at least one coupling element 56 present between the adjacent stent segments. The delivery catheter has a soft nose cone 52, a guidewire tube 64 and an outer sheath 58. A stent valve or separation element 60 disposed on the outer sheath 58 helps separate stent segments 54 selected for delivery and those remaining on the delivery catheter 62. Generally, the stent valve (separation element) is a polymeric or metallic material, although it may be made from silicone or urethane, and is soft, compliant and resilient enough to provide adequate friction. Additionally, a guidewire GW passes through the guidewire tube 64 and exits the delivery catheter from the nose cone 52.

The stent valve or separation element 60 may be mounted to the interior of sheath 58 and may be spaced proximally from the distal end of sheath 58 a distance equal to the length of about ½ to 1 stent segments. Stent valve or separation element 60 may comprises an annular ridge configured to frictionally engage stent segments 54 to facilitate control of the spacing between those segments to be deployed distally of sheath 58 and those to be retained within sheath 58. Stent valve 50 may also comprise any of the structures described in U.S. patent application Ser. No. 10/412,714 filed Apr. 10, 2003 (U.S. Pat. Pub. No. 2004/0093061 A1), which is incorporated herein by reference.

In FIG. 2B, stent segments 55 are selected for deployment and exposed from the outer sheath 58 to the lesion L. With a plurality of stent segments 55 slidably positioned over expandable member or balloon 63, pusher 68 may be axially slidable relative to an inflation shaft to engage stent segments 55. In order to move stent segments 55 relative to balloon 63, pusher 68 may be pushed distally to advance stent segments 55 over expandable member or balloon 63 or pusher 68 may be held in a stationary position while expandable member 63 is drawn proximally relative to stent segments 55. When the first stent segments 55 are initially exposed for deployment, pusher 68 may not need to be actuated if the stent segments 55 are initially positioned at the distal end of expandable member 63.

In either case, sheath 58 is axially movable relative to expandable member 63, pusher 68, and stent segments 55 and sheath 58 may be repositioned proximally or distally to selectively expose a desired length of the expandable member and stent segments thereon according to the length of the lesion L to be treated. In preferred embodiments, sheath 58 has a radiopaque marker (not shown) at its distal end, and a second radiopaque marker is located near the distal end of expandable member 63, thus allowing fluoroscopic observation of the exposed length of expandable member 63 and stent segments thereon distal to sheath 58. Further details are shown and described in U.S. patent application Ser. No. 10/746,466 filed Dec. 23, 2003 (U.S. Pat. Pub. No. 2005-0149159 A1), which is incorporated herein by reference.

With the desired number of stent segments 55 selected, sheath 58 may be retracted proximally relative to expandable member 63. Stent valve 60 engages the distal most stent segment 55 within sheath 58 so that the stent segments within sheath 58 are retracted along with the sheath relative to expandable member 62. This separates stent segments 55 exposed distally of sheath 58 from stent segments 57 held within sheath 58, as illustrated in FIG. 2C. Various other aspects of the construction of delivery catheter and stent segments are described in further detail in U.S. patent application Ser. No. 10/637,713 filed Aug. 8, 2003 (U.S. Patent Publication 2004-0098081 A1), which has been incorporated above by reference.

As illustrated, the stent segments 55 positioned along the delivery catheter 62 may slide freely relative to one another prior to expansion. Because the individual stent segments 55 in their unexpanded configuration are disconnected or uncoupled from one another, the delivery catheter 62 may retain its flexibility, particularly when advanced through tortuous regions of a patient's anatomy. Moreover, the uncoupled stent segments may further facilitate the separation and release of adjacent stent segments to be expanded, as illustrated in FIG. 2C.

Although the stent segments are disconnected or uncoupled from one another, they remain aligned with respect to another such that the complementary portions of one or more coupling mechanisms 56 formed between adjacent stent segments may be engaged upon stent expansion, as described in further detail below. Stent segments may accordingly be coupled together by expansion of the balloon or other expandable member.

FIG. 2D, balloon 63 on the delivery catheter 62 is inflated, radially expanding stent segments 66. Once the balloon 63 is expanded, the expanded stent segments 66 may become secured to one another upon expansion, in part, by utilizing the one or more coupling mechanisms 56 between adjacent stent segments which securely interlock the segments to one another by taking advantage of the changing geometry of the stents 66 during expansion. The complementary portions of the coupling mechanism 61 between the expanded stent segment 66 and unexpanded stent segment 57 may be seen. With the stent segments 66 expanded against the lesion L and secured to one another, the balloon 63 may be deflated and the delivery system 50 removed from the vessel or moved to the site of another lesion and the procedure repeated.

Because the length of the deployed stent is variable depending upon the length of the lesion to be treated, the number of stent segments deployed into a vessel may be altered in situ. Thus, ease of release and deployment of stent segments adjacent to one another is desirable. However, it is further desirable to maintain a relatively secure engagement between the expanded stent segments and the vessel wall, particularly along tapered vessels or vessels with an uneven anatomy, while also minimizing or preventing migration of an expanded stent segment relative to the other expanded stent segments. Stent segments which are able to slide freely relative to one another along the deployment catheter prior to expansion may be secured to one another when expanded and/or deployed into the vessel. Securement upon expansion of the stent segments may be accomplished, in part, by utilizing one or more coupling mechanisms between adjacent stent segments which are unconnected when the stent segments are contracted and which securely interlock the segments to one another by taking advantage of the changing geometry of the stents during expansion.

One or more coupling mechanisms, e.g., three to six coupling mechanisms or more, may be present between adjacent stent segments. Moreover, the one or more coupling mechanisms may be arranged circumferentially between the adjacent stent segments in a non-uniform or uniform arrangement, such as six coupling mechanisms arranged evenly around a circumference between the adjacent stent segments. Furthermore, in any of the variations described herein, the various coupling mechanisms may be optionally coated with a radiopaque material including, but not limited to, gold, platinum, etc., to facilitate visualization of a position of the stent and coupling mechanism.

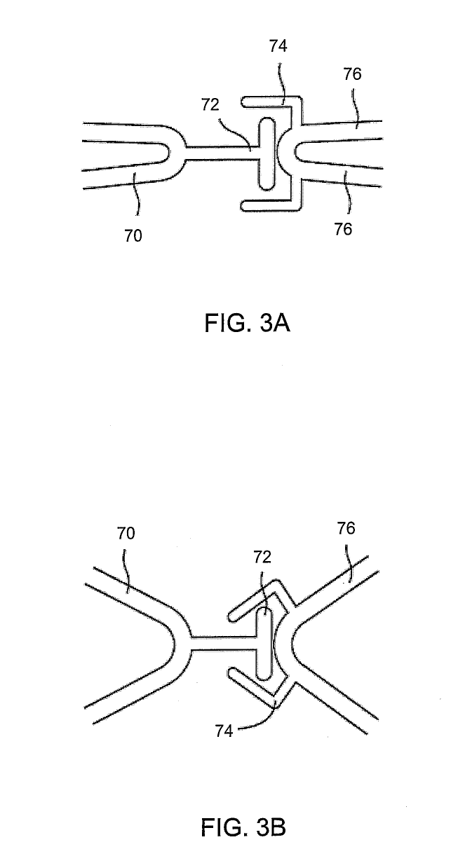

FIGS. 3A and 3B illustrate a detail view of one variation for a coupling mechanism which may be utilized to couple adjacent stent segments together as a result of changes in stent geometry and/or movement of stent struts. FIG. 3A shows a T-shaped bar 72 extending axially from stent strut 70 from a proximal stent segment and a pair of jaw members 74 extending axially from an adjacent stent segment strut 76. Bar 72 and jaw members 74 are illustrated with their respective stent segments in a low-profile constrained or undeployed configuration prior to balloon expansion. As the stent segments are radially expanded, struts 76 are deformed circumferentially away from each other, causing the set of jaw members 72 to pivot and close in around the T-shaped bar 72 capturing it and coupling the adjacent stent segments together, as illustrated in FIG. 3B.

As mentioned above, the unexpanded stent segments may freely slide relative to one another prior to their expansion and securement to one another. In alternative variations, rather than having the stent segments freely slidable and the one or more coupling mechanisms between adjacent stent segments disengaged, the coupling mechanism may be partially or loosely engaged with respect to one another. Utilizing a partial or loose engagement may allow for the delivery catheter to maintain its flexibility while also preventing or inhibiting the stent segments from misaligning and slipping from one another. Additionally, partial engagement may still allow for separation between stent segments for deployment into a vessel as the forces for maintaining the partial engagement are less than the separation forces applied by the sheath 58 and stent valve 60. Upon expansion of the underlying balloon, the coupling mechanisms between the partially engaged stent segments may be tightened or fully secured to one another.

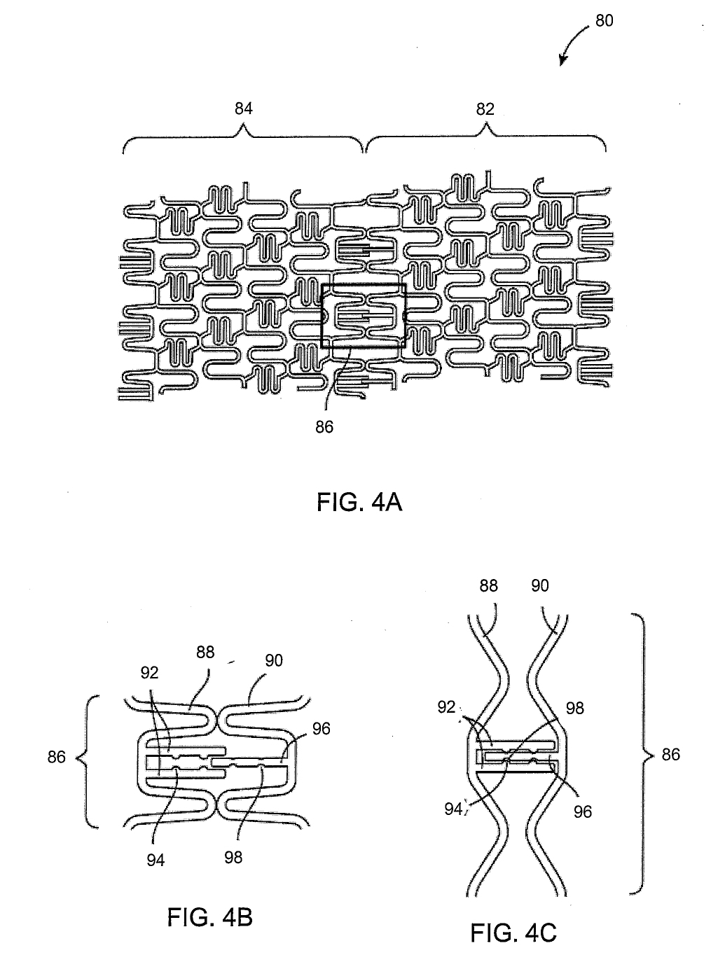

FIG. 4A illustrates another variation 80 of a coupling mechanism integrated between adjacent stent segments 82, 84 with one or more interlocking coupling mechanisms 86. Adjacent stent segments 82, 84 (shown in an unrolled and flattened two-dimensional view) each have a complementary and corresponding member of a coupling mechanism 86 that extends axially and engage one another upon stent segment expansion. Although two adjacent stent segments 82, 84 are shown in this and other examples, the number of segments is intended to be merely illustrative and is not limiting in any manner. More than two stent segments may be utilized depending upon the desired treatment and each adjacent stent segment may utilize any of the coupling mechanisms described herein or it may be omitted entirely depending upon the desired effects.

In this variation, stent struts 88 may have a pair of parallel members 92 extending axially from the proximal stent segment 84, as shown in the detail view of FIG. 4B. One or both of the parallel members 92 may define a series of protruding detents 94 along opposing lateral surfaces of members 92. The stent struts 90 on the adjacent distal stent segment 82 may also have a bar 96 extending axially towards the proximal stent segment 84 and configured to be received between parallel members 92. Bar 96 may define one or more recesses 98 on the lateral sides thereof corresponding with detents 94. In their low-profile unexpanded configuration, stent segments 82, 84 may remain unattached to one another and may thus slide freely with respect to one another. However, as the stent segments 82, 84 are expanded, as shown in FIG. 4C, the deformation of their struts 88, 90 cause parallel members 92 and bar 96 to be driven towards one another such that the complementary detents 94, 98 engage to create a closed coupling structure between adjacent stent segments 82, 84. It should be noted that parallel members 92 and bar 96 may be reversed such that parallel members 92 are on distal segment 82 pointing proximally, and bar 96 is on proximal stent segment 84 pointing distally.

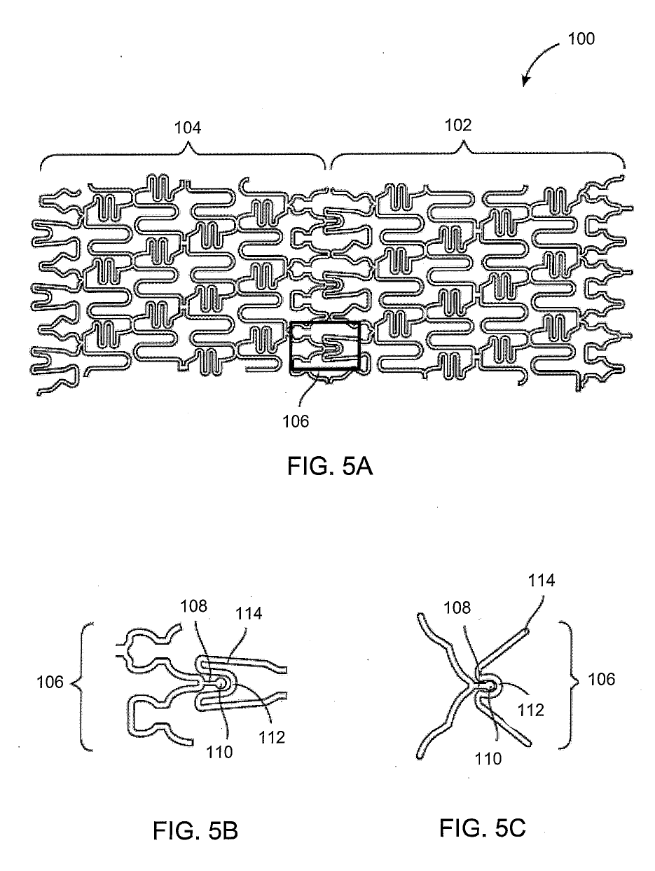

FIG. 5A shows another variation 100 of the coupling mechanism. Stent segments 102, 104 with coupling mechanism 106 that overlap or interfit with one another. A detail view of coupling mechanism 106 is shown in FIG. 5B illustrating a coupling element 108 which projects axially from proximal stent segment 104 with a rounded tip 110. Distal stent segment 102 is shown with one or more recessed regions 112 formed by the adjacent stent segment struts 114 within which coupling element 108 and rounded tip 110 may freely slide when adjacent stent segments 102, 104 are in their low-profile un-expanded delivery configurations. As the stent segments 102, 104 are expanded as illustrated in FIG. 5C, struts 114 on distal stent segment 102 move apart from one another, causing the recessed region 112 to close around and capture the tip 110, thereby linking the two adjacent stent segments 102, 104 together.

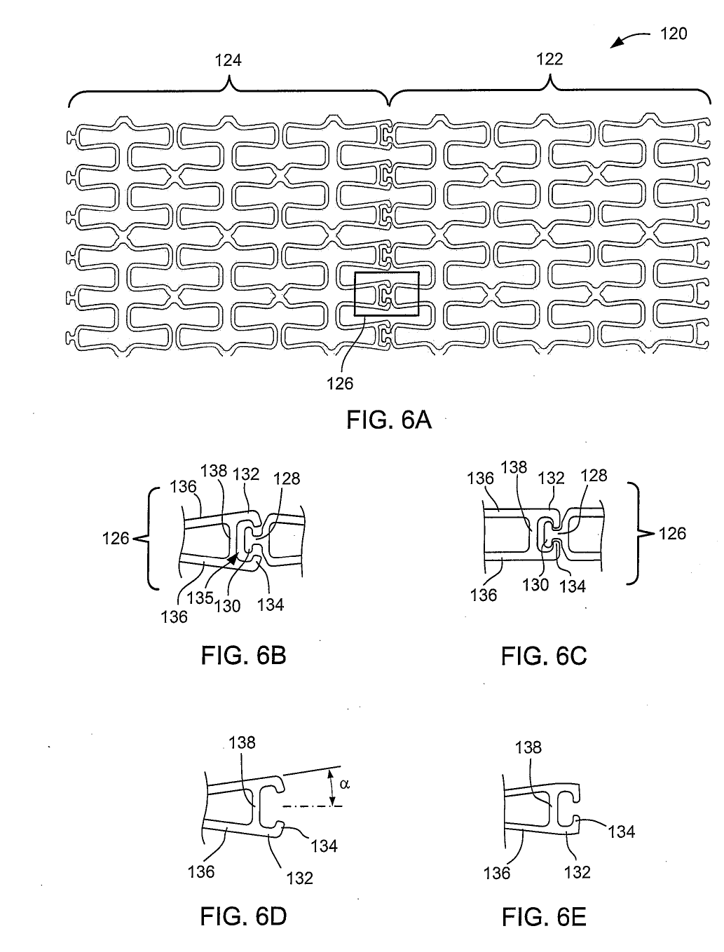

Yet another variation 120 for coupling mechanisms between adjacent stent segments is shown in stent segments 122, 124 (shown in an unrolled and flattened two-dimensional view) in FIG. 6A. As illustrated, coupling mechanisms 126 may be interdigitated between proximal stent segment 124 and distal stent segment 122 such that one or more coupling members 128 having a T-shaped tip 130 may project from stent segment 122, as shown in the detail view of FIG. 6B. Stent struts 136 on the adjacent stent segment 124 may have a pair of extending pincher or jaw members 132 which extend and terminate in a curved retaining member 134. Jaw members 132 and curved members 134 may form a receiving channel or region 135 within which coupling member 128 and T-shaped tip 130 may freely slide when adjacent stent segments 122, 124 are in their low-profile unexpanded configurations.

Upon expansion of adjacent stent segments 122,124 by the underlying balloon, stent struts 136 may be urged away from one another such that jaw members 132 and curved members 134 are pivoted about cross-member 138, which connect the proximal portions of jaw members 132 to one another. As jaw members 132 are pivoted about cross-member 138, curved members 134 are forced towards one another in a pinching motion to engage coupling member 128 and retain T-shaped tip 130 within the receiving channel, as illustrated in the detail view of FIG. 6C. The curved members 134 may be configured such that they are securely interfitted between T-shaped tip 130 and coupling member 128 to form a secure connection between adjacent stent segments 122, 124 when expanded.

Stent segments 136 and jaw members 132 may be configured in a variety of shapes provided that tip 130 may freely release from between curved members 134 when the respective stent segments are in their collapsed or low-profile configuration. To facilitate the release of tip 130, stent struts 136 and jaw members 132 may be flared with respect to a longitudinal axis of the coupling mechanism 126 such that an acute angle, α, is formed therebetween, as illustrated in FIG. 6D. Alternatively, jaw members 132 may extend parallel to one another, as shown in FIG. 6E. In either case, jaw members 132 and curved members 134 may be configured to allow for the release of coupling member 128 and tip 130 in the low-profile configuration while allowing for the secure closure of members 132, 134 upon coupling member 128 and tip 130 when stent struts are urged away from one another during expansion of the respective stent segments.

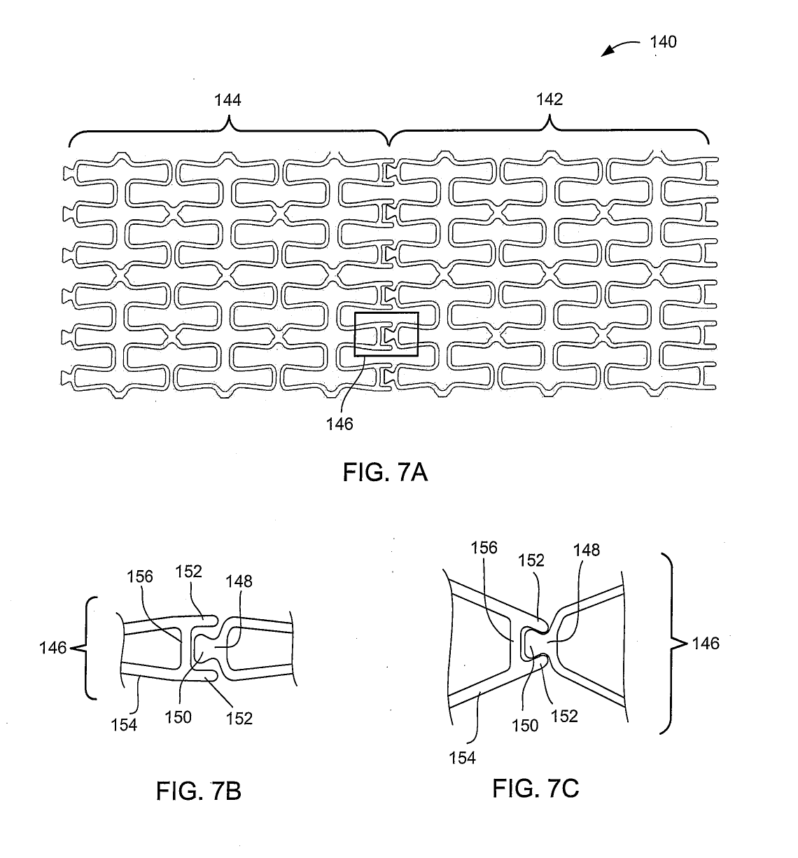

In yet another variation 140 of the coupling mechanism, FIGS. 7A to 7C illustrate stent segments 142, 144 with coupling mechanisms 146 which utilize coupling members which pinch or grasp onto an adjacent retaining tip, as shown in FIG. 7A. As above, jaw members 152 may extend from stent struts 154 with a cross-member 156 joining proximal portions of the jaw members 152 to one another and functioning as a pivot. Jaw members 152 may project from stent struts 154 as extensions which are parallel relative to one another when the stent segment is in its low-profile delivery configuration, as shown in the detail view of FIG. 7B. The adjacent coupling element is shown as a coupling member 148 projecting from the stent struts and forming a retaining tip 150 which is freely received between jaw members 152. Retaining tip 150 may be configured to have a widened distal portion such that when the respective stent segments are expanded, stent struts 154 are urged apart from one another such that jaw members 152 are pivoted about cross-member 156 and pinched upon retaining tip 150 and coupling member 148. The angle at which jaw members 152 pinch or grasp upon retaining tip 150 may be configured such that the widened tip 150 is securely retained within the angled jaw members 152, as shown in FIG. 7C.

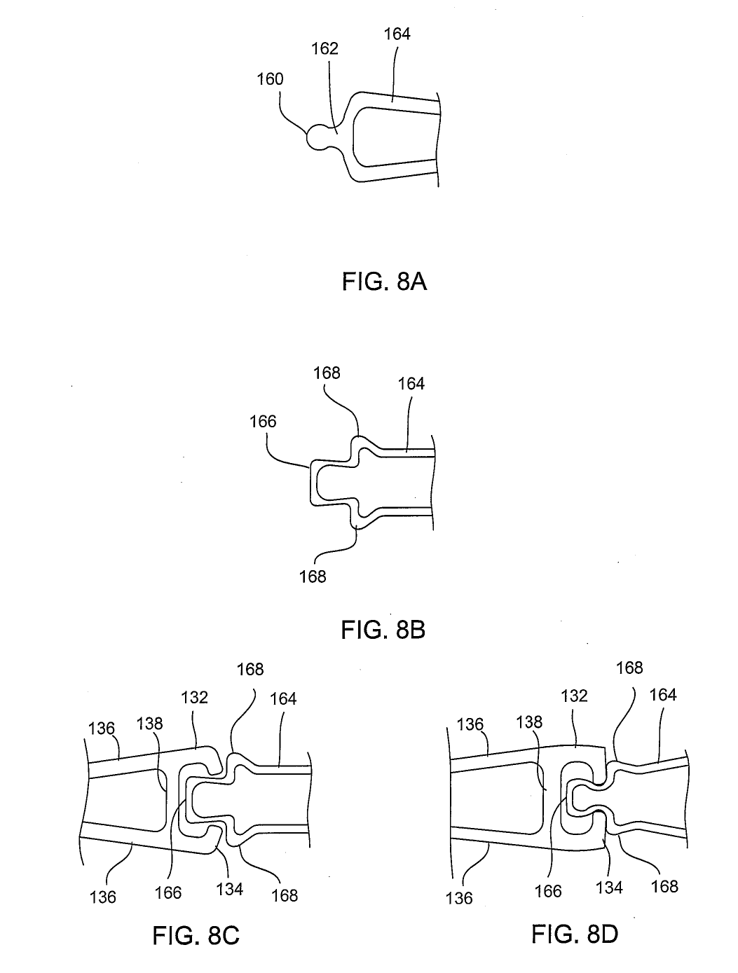

The coupling member 148 and retaining tip 150 may be configured in a variety of shapes and structures, as described herein. Additional variations for altering the retaining tip may be seen in FIG. 8A, which illustrates a detail view of a variation which may utilize a coupling member 162 having a rounded tip 160 projecting from stent struts 164. Rounded tip 160 may be configured to facilitate the entry and/or release of adjacent stent segments from one another when in their low-profile delivery configuration. Moreover, such a variation may be utilized with any of the variations of jaw members described herein, as practicable. FIG. 8B illustrates another variation where stent struts 164 may define tip 166 for coupling with its adjacent and complementary stent segments and also having one or more intra-stent cusps 168 defined along the stent struts 164. The one or more cusps 168 may define a projection or member which projects from stent struts 164 and functions as a spacer for limiting or inhibiting adjacent stent segments from colliding into one another. As shown in FIG. 8C, the one or more cusps 168 may abut against the curved members 134 to help maintain a spacing between the adjacent stent segments either when the coupling mechanism is disconnected, as shown, or when connected, as shown in FIG. 8D. Such variations as shown, as well as modifications thereof, may be utilized with any of the coupling mechanisms described herein, as practicable.

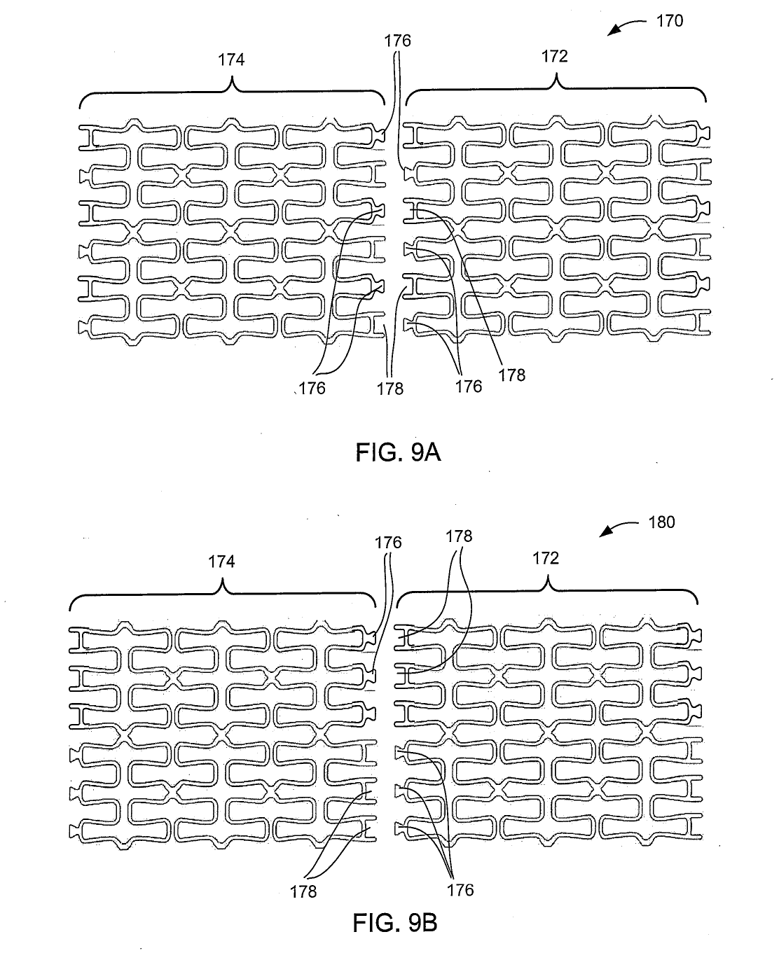

FIG. 9A illustrates yet another variation 170 for a coupling mechanism between adjacent stent segments 172, 174. The example shown illustrates the adjacent stent segments 172, 174 de-coupled or separated from one another for clarity. As shown in this variation, coupling members 176 may be alternated with jaw members 178 along the interface between each stent segments 172, 174 such that the coupling mechanisms on each respective stent segment is complementary.

Although the example shown illustrates coupling members 176 in an alternating manner with jaw members 178, these may be configured in other patterns, as illustrated by variation 180. For example, a number of jaw members 178 may be aligned in a group adjacent to another group of coupling members 176, as shown in FIG. 9B. Alternating groups of coupling members 176 and jaw members 178 may range in the number of coupling mechanisms per group as well as the alternating pattern between the groups, as practicable. In their low-profile configuration when crimped or constrained, stent segments 172, 174 may slide freely with respect to one another, but when expanded, the complementary coupling mechanisms may pinch or grasp upon one another to create a secure coupling therebetween. Additionally, as few as one coupling member may be utilized between each adjacent pair of stent segments or any suitable number of coupling members may be utilized to provide for sufficient linkage and axial strength while also providing a desired degree of lateral flexibility. Moreover, any of the variations of coupling mechanisms described herein may be utilized in either of these or other patterns, as desirable and/or practicable.

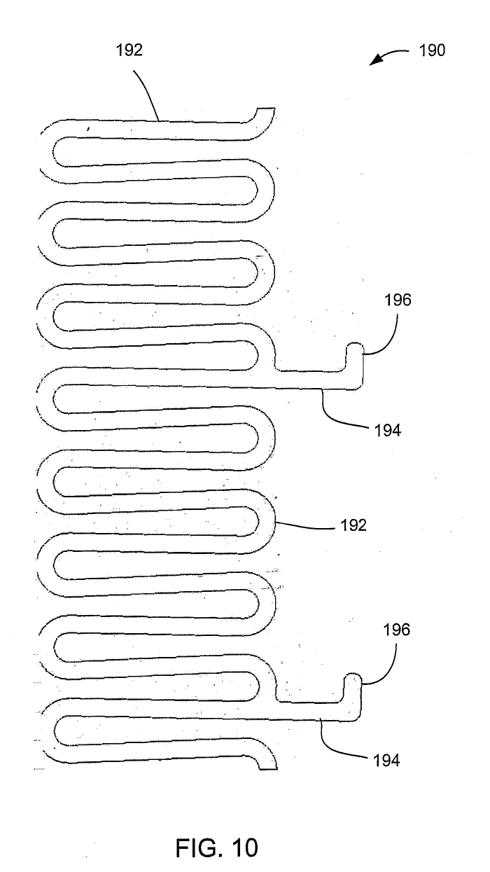

FIG. 10 shows an example of one stent segment 190 flattened to illustrate another variation where one or more single arms or coupling members 194, in this example two arms, may project from the stent struts 192 for coupling to a corresponding stent segment. The arms or coupling members 194 may project longitudinally from the stent strut 192 and curve or angle into a retaining member 196, as shown.

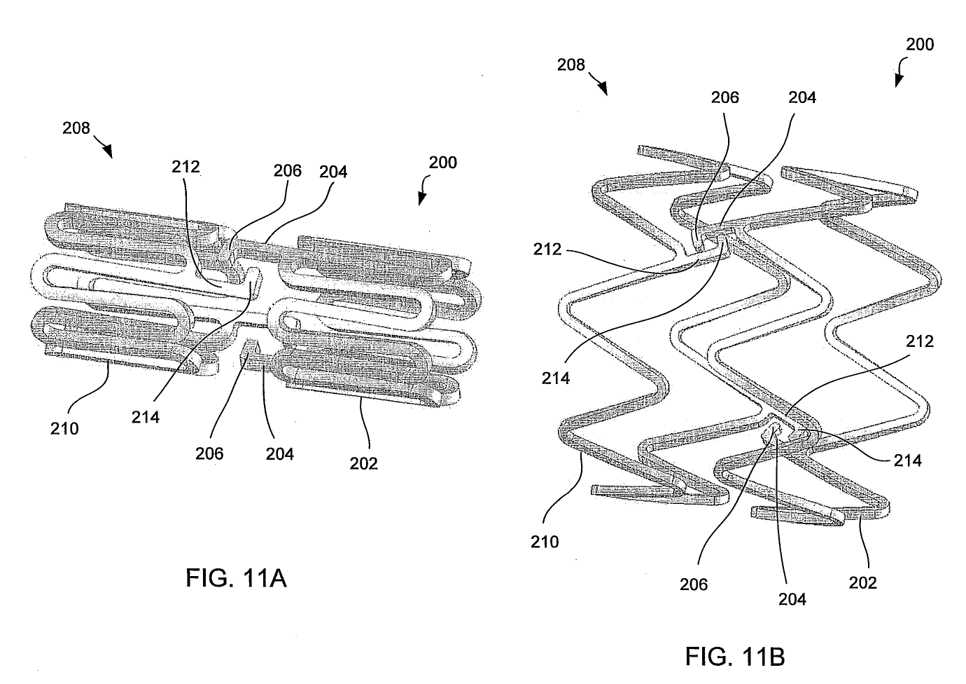

FIG. 11A shows the variation of FIG. 10 with two illustrative adjacent stent segments 200, 208 in their unexpanded and uncoupled configuration. First stent segment 200 illustrates two coupling members 204 projecting longitudinally from stent struts 202 towards adjacent stent segment 208 and with curved or angled retaining members 206 angled transversely relative to the coupling members 204. Coupling members 204 are positioned along first stent 200 such that they are located on opposite sides of the circumference facing second stent 208. Although in other variations, additional coupling members 204 may be used and positioned around the circumference of stent 200 in various uniform or non-uniform configurations.

Second stent 208 likewise has two coupling members 212 projecting longitudinally from stent struts 210 towards adjacent first stent segment 200 and with curved or angled retaining members 214 angled transversely relative to coupling members 212 and facing the direction opposite to that of retaining members 206 on the adjacent stent segment 200. With adjacent stent segments 200, 208 unexpanded, their respective coupling members may extend longitudinally such that their respective retaining members are uncoupled. When the stent segments 200, 208 are expanded, as shown in FIG. 11B, the respective stent struts 202, 210 of each stent segment 200, 208 may be urged away from one another such that the coupling members 204, 212 are rotated in opposite directions into contact against one another. Because the coupling members 204, 212 are positioned on opposite sides of each stent segment, the retaining members 206, 214 are also urged against each other and aids in locking the stent segments 200, 208 to one another to prevent any subsequent rotation or disconnection between the stent segments once expanded.

Although two coupling members are illustrated, additional members may be used and various other configurations for the retaining members may also be utilized. Moreover, the lengths of the coupling members extending between adjacent stent segments may also be varied depending upon the desired spacing between stent segments; although a relatively shortened coupling member length is generally desirable to minimize gaps between stent segments and also to facilitate scaffolding of the stent segments when expanded.

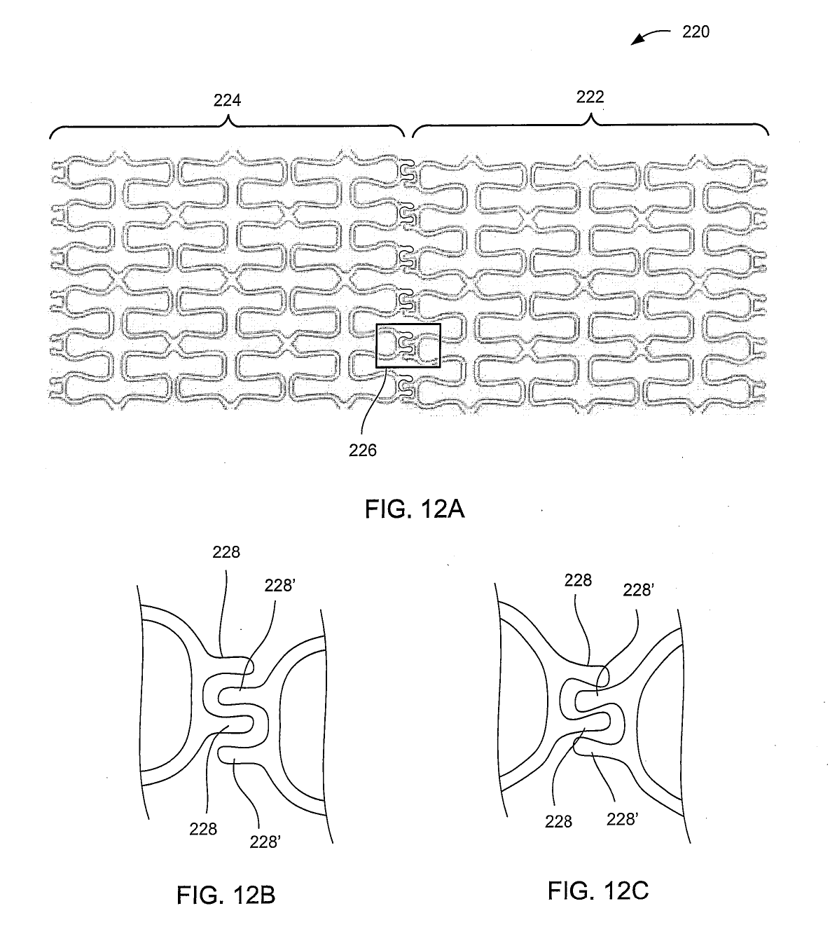

FIG. 12A illustrates another variation 220 of a coupling mechanism integrated between adjacent stent segments 222, 224 with one or more interlocking coupling mechanisms 226. Adjacent stent segments 222, 224 (shown in an unrolled and flattened two-dimensional view) each have respective pinching members 228, 228′ that extends axially and may interdigitate with one another in a disconnected or loosely coupled state, as shown in the detail view of FIG. 12B. Although two adjacent stent segments 222, 224 are shown, the number of segments may of course number fewer than two or more than two stent segments depending upon the desired effects.

Pinching members 228, 228′ may extend axially in parallel or at an angle from their respective stent struts. As the stent segments 222, 224 are expanded, as shown in FIG. 12C, the deformation of their struts cause pinching members 228, 228′ to be driven towards one another such that they engage to securely couple between adjacent stent segments 222, 224. Although the pinching members 228, 228′ are shown in this example as similar in configuration, other configurations may be utilized between adjacent stent segments.

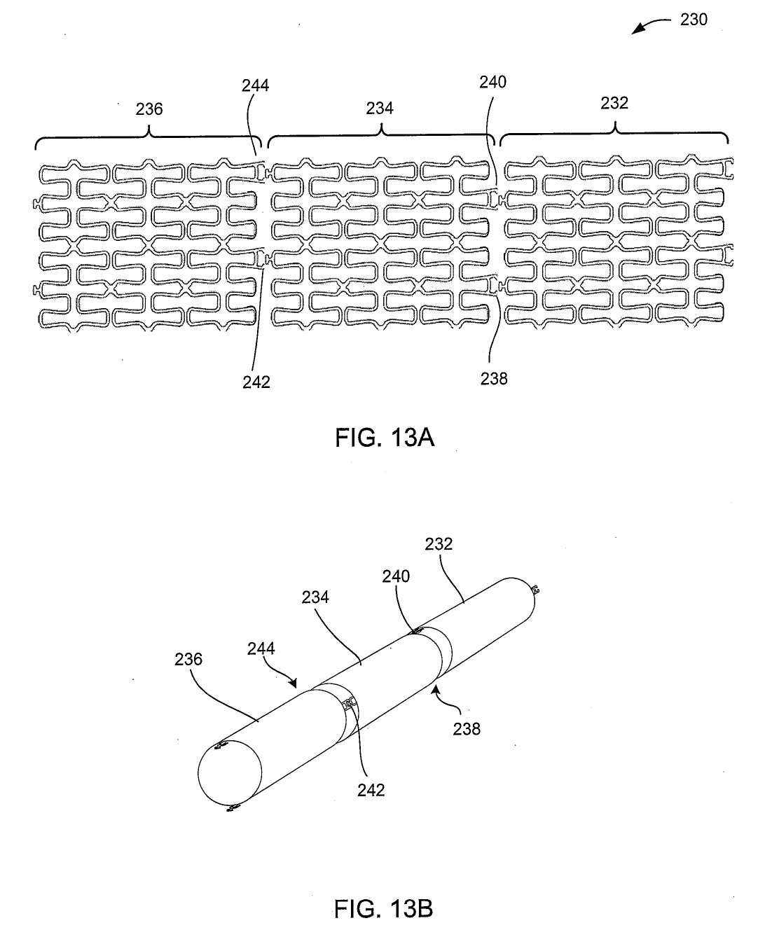

In yet other variations, stent segments may be configured such that the coupling mechanisms between adjacent segments are positioned in an alternating manner to enhance the flexibility of the deployed stent segments. An example is illustrated in FIG. 13A, which shows stent assembly 230 having adjacent stent segments 232, 234, 236 with respective coupling mechanisms aligned in alternating pairs. Coupling mechanisms 238, 240 are positioned approximately 180° from one another about the circumferences of adjacent stent segments 232, 234. Likewise, coupling mechanisms 242, 244 are also positioned approximately 180° from one another about the circumferences of adjacent stent segments 234, 236. Each respective pair of coupling mechanisms 238, 240 and 242, 244 may be positioned between adjacent stent segments to be approximately 90° from one another with respect to a longitudinal axis of the stent segments, as illustrated in the perspective view of FIG. 13B. Each pair of coupling mechanisms may be positioned at other angles from one another depending upon the desired degree of flexibility between adjacent stent segments.

In other variations, the one or more coupling mechanisms may be asymmetrically positioned with respect to one another between adjacent stent segments. For instance, different coupling mechanisms or coupling mechanisms which are configured to impart different forces may be used around a circumference of a stent segment such that when the coupling mechanisms are tightened to secure segments to one another, the coupling mechanisms may impart a shape or force a bias to the stent assembly. Couplings along one side of a stent may be tighter than the opposite side of the stent. The tightness of various couplings and the degree of bias imparted to the stent assembly may be varied depending upon the desired results.

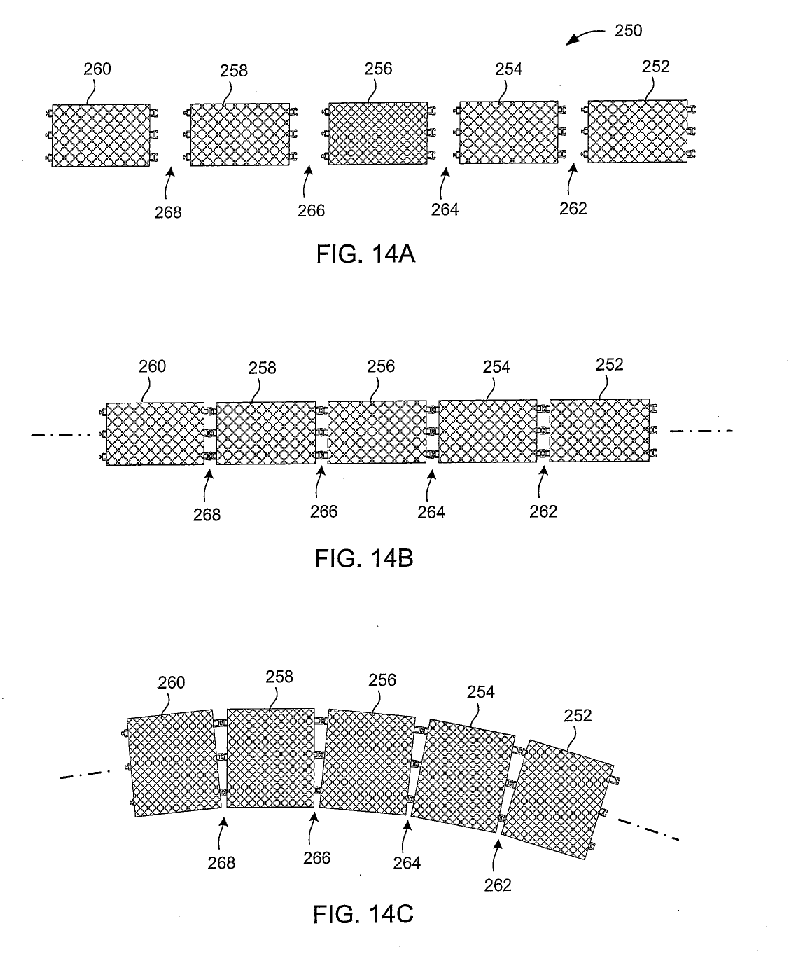

An example is illustrated in FIGS. 14A to 14C, which show an example of one stent assembly 250 having multiple stent segments 252, 254, 256, 258, 260 which may be partially coupled or free to slide with respect to one another in their unexpanded state. FIG. 14A shows the unexpanded stent segments separated from one another to illustrate the coupling mechanisms 262, 264, 266, 268 as being disengaged or uncoupled to one another and FIG. 14B shows the stent segments with the coupling mechanisms aligned to one another but uncoupled. Upon expansion of the stent segments, the coupling mechanisms may be tightened to securely engage the adjacent segments to one another, as described above. The coupling mechanisms along the coupled stent segments may be configured to engage such that the stent segments are biased or a shape is imparted to the segments, e.g., a non-straight shape such as a curve as illustrated in FIG. 14C, by the coupling mechanisms engaging at differing angles or allowing different degrees of axial separation between adjacent stent segments. Other curvatures or angles may be imparted to the adjacent stent segments depending upon the desired shape of the stent segments and vessel anatomy. Moreover, the resulting curvature or angles of the coupled stent segments may take shape due to the forces imparted by the engagement of the coupling mechanisms and in the absence of any external forces or constraints placed upon the stent segments. In an exemplary embodiment, when the stent segments are expanded, the coupling mechanisms on one side of the stent segments may allow greater axial separation or spacing between segments than those on another side of the stent segments, so that when expanded, the stent segments are drawn closer together on one side than on the other side, thereby imparting a curve to the overall stent assembly or allowing the stent assembly to assume a curved shape if implanted in a curved vessel.

The applications of the devices and methods discussed above are not limited but may include any number of further configurations. Modification of the above-described assemblies and methods for carrying out the invention, combinations between different variations as practicable, and variations of aspects of the invention that are obvious to those of skill in the art are intended to be within the scope of the claims.

What is claimed is:

1. A coupling apparatus for luminal prosthesis, comprising:
at least one coupling member extending from a first segment of the prosthesis; and
at least one receiving portion defined along a second segment of the prosthesis which is adjacent to the first segment, the first and second segments being radially expandable from a collapsed configuration to an expanded configuration,
wherein the receiving portion is in apposition to the coupling member such that the receiving portion is separable from the coupling member in the collapsed configuration and secured to the coupling member in the expanded configuration.

2. The apparatus of claim 1 further comprising a delivery catheter upon which the luminal prosthesis is delivered intravascularly.

3. The apparatus of claim 2 wherein the delivery catheter comprises an outer sheath positionable over the luminal prosthesis.

4. The apparatus of claim 3 further comprising a separation element on the delivery catheter adapted to separate a first stent segment to be retained on the delivery catheter from a second stent segment to be deployed.

5. The apparatus of claim 2 wherein the delivery catheter comprises an inflatable balloon upon which the first and second stent segments are positionable for expansion against a luminal wall.

6. The apparatus of claim 1 further comprising at least one additional coupling member extending from the first stent segment, wherein the additional coupling member is circumferentially spaced apart from the first stent segment.

7. The apparatus of claim 6 further comprising at least one additional receiving portions defined around a circumference of the second stent segment whereby each additional receiving portion is in apposition to each additional coupling member.

8. The apparatus of claim 1 wherein the coupling member extends axially from the first stent segment towards the adjacent second stent segment.

9. The apparatus of claim 1 wherein the coupling member comprises an axially projecting member having a distal tip adapted to engage the receiving portion in a secure manner.

10. The apparatus of claim 1 wherein the coupling member comprises a T-shaped projection.

11. The apparatus of claim 1 wherein the coupling member further comprises at least one intra-stent cusp adapted to maintain spacing between the first and second stent segments.

12. The apparatus of claim 1 wherein the receiving portion comprises a pair of jaw members extending from respective stent struts, wherein expansion of the luminal prosthesis urges the respective stent struts away from one another such that the jaw members close upon the coupling member.

13. The apparatus of claim 12 wherein each jaw member further comprises a curved member extending towards a longitudinal axis of the receiving portion.

14. The apparatus of claim 12 further comprising a cross-member connecting a proximal portion of the pair of jaw members.

15. The apparatus of claim 1 further comprising at least one additional receiving portion defined along the first stent segment adjacent to the at least one coupling member.

16. The apparatus of claim 15 further comprising at least one additional coupling member extending from the second stent segment adjacent to the at least one receiving portion, wherein the first and second stent segments are aligned such that each coupling member and each receiving portion are in apposition to one another in complementary alignment.

17. The apparatus of claim 1 wherein the first and second segments are disconnected from one another in the collapsed configuration.

18. The apparatus of claim 1 further comprising at least one additional segment of the prosthesis.

19. The apparatus of claim 18 wherein the additional segment is radially expandable from the collapsed configuration to the expanded configuration.

20. The apparatus of claim 19 wherein the additional segment is disconnected from the first and second segments in the collapsed configuration and connected to either the first or second segment in the expanded configuration.

21. The apparatus of claim 1 wherein at least one segment of the prosthesis carries a therapeutic agent adapted to be released therefrom.

22. The apparatus of claim 21 wherein the therapeutic agent is selected from the group consisting of antibiotics, thrombolytics, anti-thrombotics, anti-inflammatories, cytotoxic agents, anti-proliferative agents, anti-restenosis agents, endothelial cell attractors and promoters, vasodilators, gene therapy agents, radioactive agents, immunosuppressants, chemotherapeutics, stem cells, and combinations thereof.

23. The apparatus of claim 1 wherein the first and second segments of the luminal prosthesis define a non-straight shape when the coupling member and receiving portion are secured to one another in the expanded configuration in the absence of external forces or constraints.

24. The apparatus of claim 23 wherein the luminal prosthesis defines a curved shape.

25. The apparatus of claim 23 wherein the first stent segment has first and second coupling members along a first end thereof, the first and second coupling members being circumferentially spaced apart from each other, and the second stent segment defines at least first and second complementary receiving portions along a second end which is in apposition to the first end, the first and second coupling members being captured in the receiving portions upon expansion of the first and second stent segments, wherein the first coupling member and the first receiving portion allow more axial separation between the first and second ends than the second coupling member and the second receiving portion when the first and second stent segments are expanded.

26. The apparatus of claim 1 wherein a length of at least one segment of the prosthesis is less than or equal to 7 mm.

27. A luminal prosthesis, comprising:
a first tubular stent segment; and
a second tubular stent segment adjacent to and axially separable from the first stent segment,
wherein the first and second stent segments are configured to connect to one another upon radial expansion of the first and second stent segments so as to limit axial separation therebetween.

28. The luminal prosthesis of claim 27 wherein the first stent segment has at least one coupling member along a first end and the second stent segment defines at least one complementary receiving portion along a second end which is in apposition to the first end.

29. The luminal prosthesis of claim 27 further comprising a delivery catheter upon which the first and second stent segments are delivered intravascularly.

30. The luminal prosthesis of claim 29 wherein the delivery catheter comprises an outer sheath positionable over the first and second stent segments.

31. The luminal prosthesis of claim 30 further comprising a separation element positioned upon the outer sheath and movable therewith, wherein the separation element is adapted to contact and separate a first stent segment within the sheath from a second stent segment exposed outside the sheath.

32. The luminal prosthesis of claim 27 further comprising an inflatable balloon upon which the first and second stent segments are positionable for expansion against a luminal wall.

33. The luminal prosthesis of claim 28 further comprising at least one additional coupling members extending from the first stent segment.

34. The luminal prosthesis of claim 33 further comprising at least one additional receiving portion defined around the second circumference whereby each additional receiving portion is in apposition to each additional coupling member.

35. The luminal prosthesis of claim 28 wherein the coupling member extends axially from the first stent segment towards the adjacent second stent segment.

36. The luminal prosthesis of claim 28 wherein the coupling member comprises an axially projecting member having a distal tip adapted to engage the receiving portion in a secure manner.

37. The luminal prosthesis of claim 28 wherein the coupling member comprises a T-shaped projection.

38. The luminal prosthesis of claim 28 wherein the coupling member further comprises at least one intra-stent cusp adapted to maintain spacing between the first and second stent segments.

39. The luminal prosthesis of claim 28 wherein the receiving portion comprises a pair of jaw members extending from respective stent struts, wherein expansion of the second stent segment urges the respective stent struts away from one another such that the jaw members close upon the coupling member.

40. The luminal prosthesis of claim 39 wherein each jaw member further comprises a curved member extending towards a longitudinal axis of the receiving portion.

41. The luminal prosthesis of claim 39 further comprising a cross-member connecting a proximal portion of the pair of jaw members.

42. The luminal prosthesis of claim 27 wherein the luminal prosthesis defines a curved or angular shape upon radial expansion of the first and second stent segments when constrained.

43. The luminal prosthesis of claim 42 wherein the first stent segment has first and second coupling members along a first end thereof, the first and second coupling members being circumferentially spaced apart from each other, and the second stent segment defines at least first and second complementary receiving portions along a second end which is in apposition to the first end, the first and second coupling members being captured in the receiving portions upon expansion of the first and second stent segments, wherein the first coupling member and the first receiving portion allow more axial separation between the first and second ends than the second coupling member and the second receiving portion when the first and second stent segments are expanded.

44. A method for coupling two or more stent segments in a vessel lumen, comprising:
delivering at least first and second stent segments to the vessel lumen, the first and second stent segments being carried by a delivery catheter, the first and second stent segments being axially separable from one another in the delivery catheter; and
expanding the first and second stent segments in the vessel lumen such that adjacent portions of the first and second stent segments become secured to one another when expanded thereby limiting axial separation therebetween.

45. The method of claim 44 further comprising advancing at least the first and second stent segments intravascularly through the vessel lumen prior to expanding.

46. The method of claim 44 wherein delivering comprises advancing a delivery catheter upon which the first and second stent segments are positioned.

47. The method of claim 46 further comprising retracting an outer sheath to expose at least the first and second stent segments to the vessel lumen.

48. The method of claim 47 further comprising engaging additional stent segments within the outer sheath to separate the additional stent segments from the first and second stent segments as the outer sheath is retracted.

49. The method of claim 44 wherein expanding comprises expanding an inflatable balloon upon which the first and second stent segments are positioned upon.

50. The method of claim 44 further comprising selecting the first and second stent segments for deployment from three or more stent segments on the delivery catheter.

51. The method of claim 44 wherein expanding comprises expanding the first and second stent segments against a lesion.

52. The method of claim 44 wherein expanding comprises inflating a balloon to expand the first and second stent segments within the vessel lumen.

53. The method of claim 44 wherein expanding comprises urging one or more stent struts away from one another along the second stent segment adjacent to the first stent segment such that a pair of jaw members close upon a complementary coupling member extending from the first stent segment.

54. The method of claim 44 wherein expanding comprises securing a first circumference of the first stent segment to a second circumference of the second stent segments, wherein the first and second circumferences are adjacent to one another.

55. The method of claim 44 wherein delivering further comprises delivering at least a third stent segment with the first and second stent segments in the delivery catheter, the third stent segment being axially separable from the first and second stent segments.

56. The method of claim 55 further comprising separating the third stent segment from the first and second stent segments prior to expanding the first and second stent segments in the vessel lumen, the third segment being retained in the delivery catheter.

57. The method of claim 44 wherein delivering comprises delivering at least first and second stent segments in an artery of a patient.

58. The method of claim 44 further comprising eluting a therapeutic agent from at least one of the first and second stent segments in the vessel lumen.

59. The method of claim 44 wherein expanding comprises expanding at least the first and second stent segments while retaining at least one additional stent segment on the delivery catheter.

60. A system for deploying a luminal prosthesis, comprising:
a delivery catheter;
at least two tubular stent segments positioned along the delivery catheter,
wherein the tubular stent segments are disconnected from one another and axially separable in a collapsed configuration, and
wherein the tubular stent segments are configured to connect to one another upon radial expansion of the stent segments into an expanded configuration so as to limit the axial separation thereof.

61. The system of claim 60 further comprising an outer sheath positionable over the stent segments.

62. The system of claim 60 further comprising a separation element on the delivery catheter for axially separating the tubular stent segments.

63. The system of claim 62 wherein the separation element comprises a separation element adapted to contact and separate stent segments for deployment.

64. The system of claim 60 further comprising an inflatable balloon upon which the tubular stent segments are positionable for radial expansion.

65. The system of claim 60 further comprising at least one coupling mechanism between each adjacent stent segment, wherein the coupling mechanism is disconnected and axial separable between stent segments in the collapsed configuration.

66. The system of claim 65 wherein the at least one coupling mechanism is configured to connect upon radial expansion of the stent segments into the expanded configuration such that the stent segments are secured to one another.

67. The system of claim 60 further comprising at least one additional stent segment positioned along the delivery catheter proximal to the at least two tubular stent segments.

68. The system of claim 67 wherein the additional stent segment is disconnected from the at least two tubular stent segments in the collapsed configuration.

69. The system of claim 68 wherein the additional stent segment is retained on the delivery catheter when the tubular stent segments are radial expanded.

70. The system of claim 60 wherein at least one stent segment carries a therapeutic agent releasable therefrom.

71. A luminal prosthesis, comprising:
a first tubular stent segment; and
a second tubular stent segment adjacent to and at least partially coupled to the first stent segment,
wherein the first and second stent segments are axially separable from one another via a separating mechanism when the stent segments are in a collapsed configuration, and
wherein the first and second stent segments are configured to connect to one another upon radial expansion such that the first segment is less bendable relative to the second stent segments are stiffened.

72. The luminal prosthesis of claim 71 wherein the first stent segment has at least one coupling member along a first end and the second stent segment defines at least one complementary receiving portion along a second end which is in apposition to the first end, the coupling member being captured in the receiving portion upon expansion of the first and second stent segments.
